(19) United States
(12) Patent Application Publication
(10) Pub. No.: US 2016/0370870 A1
(43) Pub. Date: Dec. 22, 2016

(54) METHOD AND MEDICAL IMAGING APPARATUS FOR DETECTING AT LEAST ONE INPUT GESTURE OF A USER

(71) Applicant: Siemens Healthcare GmbH, Erlangen (DE)

(72) Inventors: Arne Hengerer, Moehrendorf (DE); Eva Rothgang, Nuernberg (DE)

(73) Assignee: Siemens Healthcare GmbH, Erlangen (DE)

(21) Appl. No.: 15/185,681

(22) Filed: Jun. 17, 2016

(30) Foreign Application Priority Data
Jun. 19, 2015 (DE) 102015211331.0

(51) Int. Cl. G06F 3/01; A61B 5/055; A61B 5/00; G06K 9/00

(52) U.S. Cl. CPC G06F 3/017; G06K 9/00335; A61B 5/055; A61B 5/704

(57) ABSTRACT

In a medical imaging apparatus having a medical data acquisition scanner operated by a control computer, and an operating method therefor a gesture detector in communication with the control computer detects at least one input gesture made by a user and provides an electronic signal, in response to the at least one input gesture, to the control computer that causes the control computer to control an aspect of operation of said data acquisition scanner dependent on said at least one input gesture. Detection of the at least one input gesture by the gesture detector is activated by providing an activation signal to the gesture detector from an activation status controller in response to a manual input to the activation status controller made by the user, with the gesture detector being enabled to detect the at least one input gesture only after being provided with the activation signal.

### BACKGROUND OF THE INVENTION

### Field of the Invention

The present invention concerns a method for detecting at least one input gesture of a user for operation of a medical imaging apparatus. The present invention also concerns a medical imaging apparatus having a data acquisition scanner, which is designed for detecting medical image data. The invention also concerns a user interface, which is arranged inside an examination room, and concerns an activation status controller designed for activating detection of at least one input gesture.

### Description of the Prior Art

In the case of interventions guided by medical imaging examinations, for example in the case of biopsy examinations and/or ablations on a patient, it is necessary for a user, in particular for a physician, to interact with the medical imaging apparatus. The consequence of this is that a sterile working method of the user is necessary, and a user interface of the medical imaging apparatus must also be operated. During the medical imaging, examination information is presented as an output to the user and/or information and/or parameters are entered as an input by the user via the user interface. If, for hygiene reasons, information is entered solely by an input gesture of the user, the situation can occur where a random gesture of the user is incorrectly identified as an input gesture, or an input gesture effected by the user is not recognized and/or identified as an input gesture.

### SUMMARY OF THE INVENTION

An object of the present invention is to enable simple operation for a user and clear recognition of input gestures at a user interface during a medical imaging examination.

The above object is achieved in accordance with the invention by a method for detecting at least one input gesture of a detector for operating a medical imaging apparatus, which includes detection of the at least one input gesture of the user with a gesture detector, and that also includes activating detection of the at least one input gesture by a gesture detector by placing the gesture detector in an activated state by a signal supplied thereto from an activation status controller, in response to a manual input made by the user to the activation status controller.

As used herein, activating detection of the at least one input gesture means that the activation status controller is in an activation state in which the activation status controller activates the units required for gesture detection, for example a gesture detector having a camera, so these are in a detection mode in which input gestures can be detected. The activation status controller preferably has at least two activation states, wherein in a first activation state the activation status controller is designed to activate all units required for detection of an input gesture of the user and to put them into a detection mode. In a second activation state the activation status controller is designed to deactivate all units required for detection of an input gesture of the user and to put them into a non-detection mode. In non-detection mode detection of an input gesture, in particular a control gesture for controlling the medical imaging apparatus, of the user is precluded, but detection of an activation gesture can still occur in the non-detection mode. The activation status controller controls the units required for gesture detection dependent on the current activation state, so these units always have a current operating mode for detecting input gestures. The units for detecting input gestures of the user preferably include a user interface that is designed for presenting output information and for receiving input information and/or parameters, and that is arranged inside the examination room in which a scanner of the medical imaging apparatus is also situated. The activation signal preferably is a signal containing information as to which activation state of the activation status controller should be set in the activation status controller.

The gesture detector is configured to detect an input gesture of the user. The gesture detector for this purpose has a detection region within which the input gesture of the user can be detected. The gesture detector is preferably incorporated in a user interface of the medical imaging apparatus. An input gesture of the user means a gesture by which an input can be effected by the user at a user interface, with the detected input gesture containing information as to operation and/or control of the medical imaging apparatus.

The user is normally a medical operator who prepares and/or supervises the pending medical imaging examination. The medical operator can be, for example, a physician carrying out treatment.

The invention enables simple operation and clear recognition of input gestures at a user interface during, for example, an interventional medical imaging examination. This has the advantageous consequence that hygiene regulations, in particular with respect to sterility, during such an interventional medical imaging examination, can be easily and reliably maintained. Furthermore, additional input devices, such as a keyboard and/or a touch display, can be foregone and laborious cleaning of such input devices thus is also avoided. Furthermore, the medical imaging examination can be carried out solely from an examination room, since operation and control of the medical imaging examination can occur in the examination room. A continual change in position between the examination room and the control room, in which system control computer and console of the medical imaging apparatus are situated, is thus advantageously avoided.

Furthermore, misinterpretation of input gestures of the user can be precluded in this way. The workflow can be simplified because information and/or parameters can be easily input in a time-saving manner by an input gesture.

The activation status controller has at least one activation state and this at least one activation state is visually depicted for the user. As used herein, an activation state means an operating state and/or an operating mode of the activation status controller. Detection of the at least one input gesture is preferably dependent on a current activation state of the activation status controller. The activation status controller preferably has at least two different activation states, with an input gesture of the user can being detectable in a first activation state, and the activation status controller is in a non-detection state in a second activation state. This embodiment of the invention enables simple detection of an activation state for the user, so the user can optionally also change this activation state.

In a further embodiment of the invention, the at least one activation state is depicted within a field of view that the user has while implementing the image data acquisition procedure.

Advantageous visibility of the at least one activation states can be achieved thereby. The visual depiction of the at least one activation state of the activation status controller can be displayed directly in a viewing region and/or working region of the user and/or a medical operator. Such a field of view means a region of the body of the patient that is to be examined by the medical imaging apparatus. Alternatively or additionally, the field of view can be a projection region of a projector of the activation status controller and/or the field of view can encompass a detection region for detecting gesture data, in particular position data of the user, by the gesture detector.

In a further embodiment of the invention, the activation status controller has at least two different activation states, with the at least two different activation states being respectively depicted by different symbols and/or different colors. This enables intuitive and simple detection of a current activation state of the activation status controller.

In another embodiment, the activation status controller has at least two different activation states, with an activation si_{g}nal for activating at least one activation state of the at least two activation states being triggered by an activation gesture within field of view. A particularly time-saving workflow for changing the activation state of the activation status controller, for example, from a non-detection state into a detection state can be achieved, thereby. The activation state of the activation status controller is preferably activated and/or triggered by the activation gesture within the field of view by the at least one activation gesture can detected. The activation gesture can be, for example, the user placing a hand in the field of view. Any other activation gesture that is considered reasonable to those skilled in the art is suitable for triggering the at least one activation state.

In another embodiment, the activation status controller has at least two different activation states, with an activation signal for activating at least ones activation state of the at least two activation states being triggered by actuation of at least one operating element. This can prevent misinterpretation of input gestures. The operating element can be any type of operating element considered reasonable to those skilled in the art, such as a button and/or an operating panel. The operating element is particularly advantageously formed by a pedal, which is operated by the user's foot, so the workflow of the user does not have to be interrupted.

The invention also encompasses a medical imaging apparatus having an image data acquisition scanner, which is designed for detection of raw data that are converted into medical image data, a user interface which is situated inside an examination room, and an activation status controller, wherein the medical imaging apparatus is designed to implement the method for detecting at least one input gesture of a user for operating the medical imaging as described above. In this apparatus, a gesture detector is provided that is configured to detect at least one input gesture of the user which serves an input command to set at least one aspect of operation of the scanner. The gesture detector, in addition to being in communication with the control computer for the scanner, is in communication with an activation status controller. The activation status controller is configured to activate the gesture detector, so as to enable the gesture detector to detect the input gesture, in response to a manual input made by the user to the activation status controller.

The apparatus according to the invention enables simple operation and clear recognition of input gestures at a user interface during, for example, an interventional medical imaging examination. The advantageous consequence of this is that hygiene regulations, in particular with respect to sterility, during, for example, an interventional medical imaging examination, can also be easily and reliably maintained. Furthermore, additional input devices, such as, for example, a keyboard and/or a touch display, are advantageously not needed and so laborious cleaning of such input devices is avoided. Furthermore, the medical imaging examination can be carried out solely from an examination room, since operation and control of the medical imaging examination can occur in the examination room. Continually changing position between the examination room and a control room, in which a system control computer and console of the medical imaging apparatus are situated, is thereby avoided.

In addition, misinterpretation of input gestures of the user can be avoided in this way. The workflow can also be simplified because information and/or parameters can be entered simply and in a time-saving manner by an input gesture.

The advantages of the inventive medical imaging apparatus substantially correspond to the advantages of the inventive method for detecting at least one input gesture as described above in detail. Features, advantages or alternative embodiments mentioned in connection with the method are applicable to the apparatus as well.

When the user interface has a gesture detector, simple and fast detection of an input gesture of the user can advantageously be achieved. The gesture detector preferably has a camera for this purpose.

In an embodiment of the inventive apparatus, the user interface has at least one manual operating element for activating detection of at least one input gesture. Misinterpretation of input gestures can advantageously be prevented thereby, since the gesture recognition has to be activated first and therefore random gestures of the user are not detected. The operating element is particularly advantageously formed by a pedal that is operated by the user's foot, so the workflow of the user does not have to be interrupted.

In a further embodiment of the invention the user interface has a depiction unit, so a current activation state for detection of input gestures can be depicted for the user. The depiction unit preferably is a projector since information, in particular activation information, can be directly projected into a viewing region and/or a working region of the user thereby. The projector is designed to project the activation information into a field of view that the user can see during the image data acquisition procedure.

The invention also encompasses a non-transitory, computer-readable data storage medium encoded with program code, the storage medium being loaded directly into a memory of a programmable system control computer of a medical imaging apparatus. The program code causes the control computer to implement the method for detecting at least one input gesture in accordance with the invention, when the program code is executed in the system control computer of the medical imaging apparatus. The program code may need peripheral support, such as libraries and help functions, in order to implement the corresponding embodiments of the method. The program code may be a source code, which has still to be compiled and embedded or that only has to be interpreted, or an executable software code that for execution then only has to be loaded in the corresponding processor.

### BRIEF DESCRIPTION OF THE DRAWINGS

FIG. 1 schematically illustrates an inventive medical imaging apparatus in accordance with the invention.

FIG. 2 is a flowchart of an inventive method for detecting at least one input gesture of a user.

FIG. 3 shows a first exemplary embodiment for depicting an activation state of an activation status controller.

FIG. 4 shows a second exemplary embodiment for depicting an activation state of an activation status controller.

### DESCRIPTION OF THE PREFERRED EMBODIMENTS

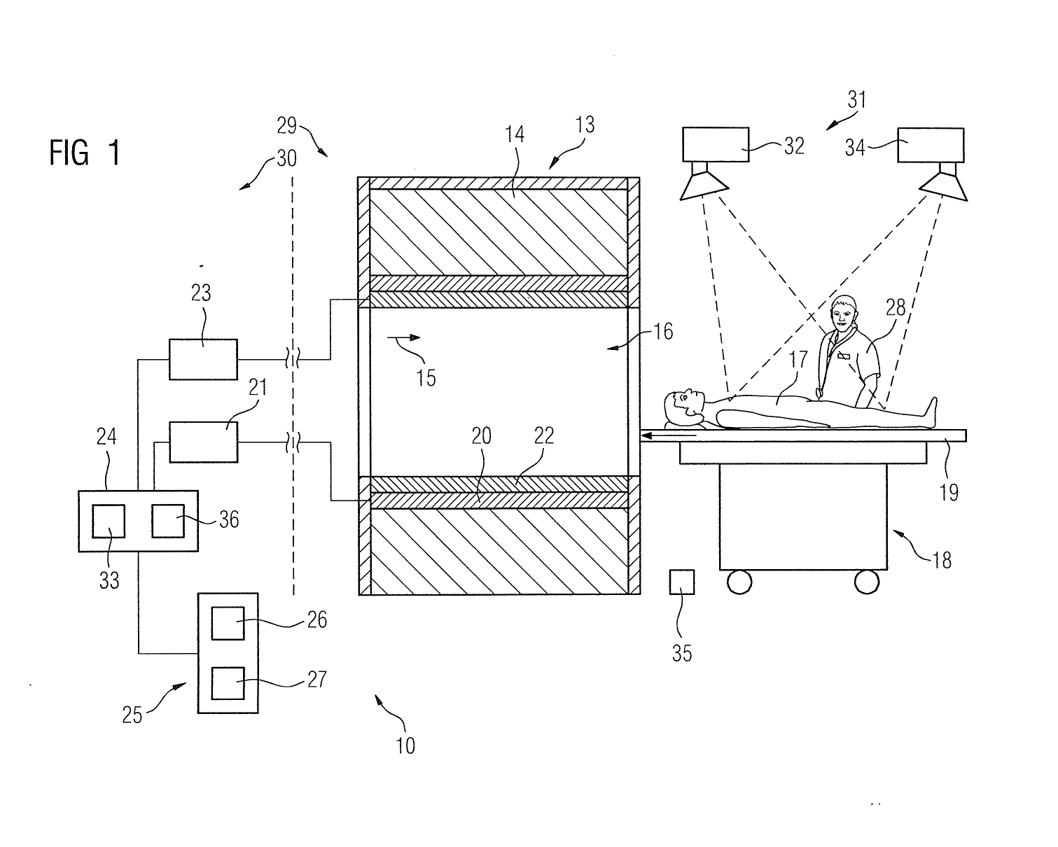

FIG. 1 schematically shows a medical imaging apparatus 10. In the exemplary embodiment, the medical imaging apparatus 10 is formed as a magnetic resonance apparatus 11. The design of the medical imaging apparatus 10 is not limited to a magnetic resonance apparatus 11. The medical imaging apparatus 10 can be formed by any medical imaging apparatus that is considered expedient to those skilled in the art, such as a computed tomography apparatus, a positron emission tomography apparatus, etc.

The magnetic resonance apparatus 11 has a scanner 13, formed by a magnetic unit. The scanner 13 has a superconductive basic field magnet 14 for generating a strong and constant basic magnetic field 15. The magnetic resonance scanner 13 has a patient-receiving region 16 for receiving a patient 17. In the exemplary embodiment the patient-receiving region 16 is cylindrical and is cylindrically surrounded in the circumferential direction by the scanner 13. A different design of the patient-receiving region 16 is also conceivable. The patient 17 can be moved by a patient-positioning device 18 of the magnetic resonance device 11 into the patient-receiving region 16. The patient-positioning device 18 has for this purpose an examination table 19 designed to move within the patient-receiving region 16.

The scanner 13 also has a gradient coil arrangement 20 for generating magnetic field gradients, which are used for spatial encoding during data acquisition. The gradient coil arrangement 20 is controlled by a gradient control processor 21 of the magnetic resonance apparatus. The scanner 13 also has a radio-frequency antenna 22. The radio-frequency antenna 22 is controlled by a radio-frequency antenna control processor 23 of the magnetic resonance apparatus so as to irradiate radio-frequency pulse sequences into the patient-receiving region 15 of the scanner 13 so as to cause nuclear spins in the patient 17 to deviate from the polarization produced by the basic field 15.

For controlling the basic field magnet 14, the gradient control processor 21, and for controlling the radio-frequency antenna control processor 23, the magnetic resonance apparatus has a system control computer 24. The system control computer 24 centrally controls the magnetic resonance apparatus, such as to execute a predetermined imaging gradient echo sequence. Furthermore, the system control computer 24 has an evaluation processor (not shown) for evaluation of medical image data acquired during the magnetic resonance examination. The magnetic resonance apparatus also has a control console 25 connected to the system control computer 24. Control information, such as imaging parameters, and reconstructed magnetic resonance images, can be displayed on a display unit 26, for example on at least one monitor, of the control console 25 for a user 28, such as a medical operator. The control console 25 also has an input unit 27 via which information and/or parameters can be entered by the medical operator during a scanning process.

The system control computer 24 is situated together with the operating console 25, gradient control processor 21 and radio-frequency antenna control processor 23 inside a control room 30. The scanner 13 is situated inside an examination room 29, with the examination room 29 preferably being shielded outwardly and inwardly in order to shield against disruptive effects that can affect a medical imaging examination and that may be caused by the medical imaging apparatus 10. In particular, the examination room 29, in one embodiment of the medical imaging apparatus 10 as a magnetic resonance apparatus, is shielded with respect of magnetic fields and against electromagnetic radiation, in particular radio-frequency radiation. Preferably all units and modules for controlling the medical imaging apparatus 10 are arranged in the control room 30. The control room 20 is designed separately and so as to be shielded from the examination room 29 with respect to an exchange of electromagnetic radiation and/or magnetic fields.

For exchange of information between the magnetic resonance apparatus and the user 28 within the examination room 29 and/or for controlling and/or monitoring the magnetic resonance apparatus and/or a magnetic resonance examination, the magnetic resonance apparatus has a user interface 31 that is situated inside the examination room 29. For detection of input gestures of the user 28, the user interface 31 has a gesture detector 32. In the exemplary embodiment, the gesture detector 32 has a camera, by which a position and/or movements of the user 28 can be detected. The position data and/or movement data detected by the gesture detector 32 are transmitted via a data transmission bus (not shown) to a gesture evaluation processor 36 of the system control computer 24 and are evaluated therein. The gesture detector 32 is situated inside the examination room 29; the gesture evaluation processor 36 is situated inside the control room 30.

The user interface 31 also has a depiction unit 34 formed by a projector which is provided for projection of information and/or data within the examination room 29.

To avoid misinterpretation of individual input gestures of the user 28, the user interface 31 also has an activation status controller 33 by which detection of input gestures by means of the gesture detector 32 is activated. In the present exemplary embodiment the activation status controller 33 is incorporated by the system control computer 24 and is arranged inside the control room 30. Gesture detection is activated by the gesture detector 32 by the activation status controller 33.

An activation signal for activating detection of at least one input gesture of the user 28 can be detected by the depictor 34, in particular the projector, in collaboration with the gesture detector 32, with detection of the activation signal being controlled by the activation status controller 33.

Furthermore, the user interface 31 has an additional operating element 35, which in the exemplary embodiment is formed by a pedal. With this operating element 35, an activation signal can likewise be entered, with detection of the activation signal by the operating element 35, in particular the pedal, being controlled by the activation status controller 33 here as well.

In an alternative embodiment of the invention, the activation status controller 33 is designed without the operating element 35. Furthermore, it is also possible for the activation status controller 35 to have only the operating element 35 available for activating detection of at least one input gesture of the user 28 and for the activation status controller 33 to not have a projector.

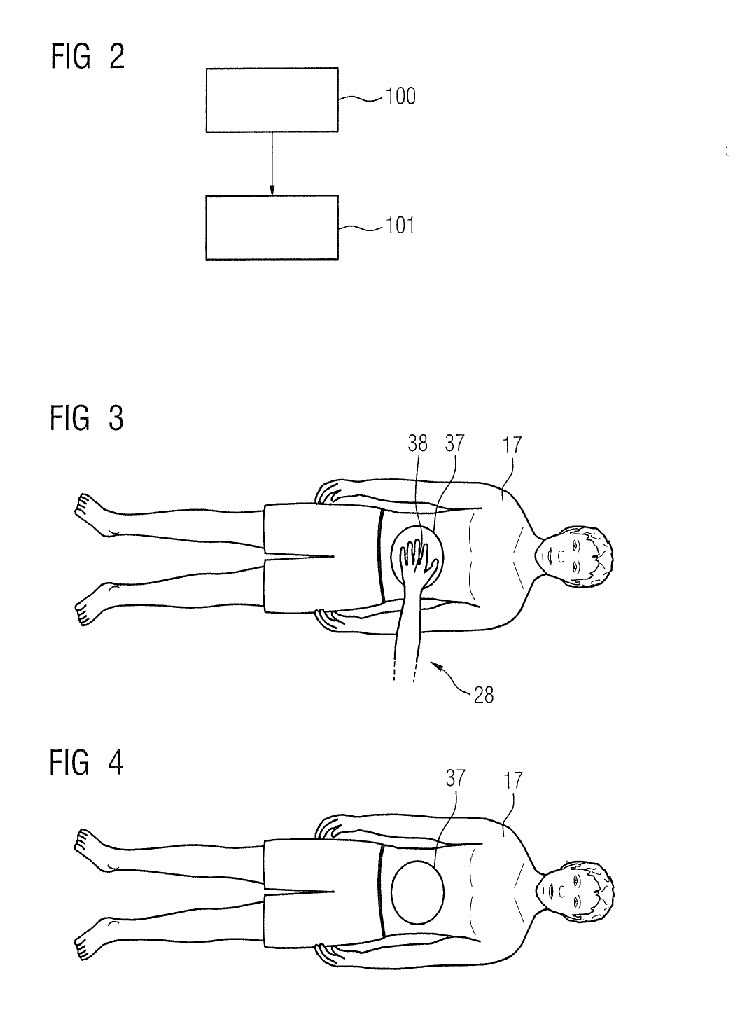

FIG. 2 shows an embodiment of the inventive method for detecting at least one input gesture of the user 28 for operating the medical imaging apparatus 10, in particular the magnetic resonance apparatus, during the medical imaging examination, in particular a magnetic resonance examination. This method is implemented by the activation status controller 33 in collaboration with the user interface 31. For this purpose, the activation status controller 33 has appropriate software and/or computer programs that are stored in a memory (not shown) of the system control computer 24, For implementing the method, the software and/or computer programs is/are run by a processor (not shown) of the system control computer 24.

At the beginning of the method, detection of the at least one input gesture of the user 28 is activated by the activation status controller 33 in a first method step 100. The activation status controller 33 for this purpose has two activation states. In a first activation state of the two activation states the activation status controller 33 controls the gesture detector 32 such that the gesture detector 32 is in a detection mode. This detection mode of the gesture detector 32 puts the gesture detector 32 in an operating state in which the gesture detector is active. Furthermore, in the first activation state of the activation status controller 33, the gesture detector 32 is also designed to detect activation gestures.

In a second activation state of the two activation states of the activation status controller 33, the gesture detector 32 is controlled by the activation status controller 33 so as to put the gesture detector 32 is in a non-detection mode. In this non-detection mode, the gesture detector 32 is not enabled for detection of an input gesture, for example a control gesture for controlling the magnetic resonance apparatus. In this non-detection mode, the gesture detector 32, and therewith input gesture detection, is inactive. In other words, no input gesture of the user 28 can be detected. In this second activation state of the activation status controller 33, the gesture detector 32 is configured solely to detect activation gestures.

At the beginning of the method, the activation status controller 33 is in the second activation state in which the gesture detector 32 is also in non-detection mode.

A current activation state of the activation status controller 33, and therewith a current operating mode of the gesture detector 32, is visually depicted and/or displayed for the user 28 by means of the depiction unit 34. For this purpose, the current activation state of the activation status controller 33 is depicted in a field of view. The field of view preferably encompasses a region of the patient 17 to be examined and/or is situated inside a working region and/or a viewing direction of the user 28. Alternatively or additionally, the field of view can encompass a depiction region of the depiction unit 34 and/or a detection region of the gesture detector 32. In the present exemplary embodiment the measuring field comprises a central and/or middle region of a surface of the body of the patient 17. The activation state in the central and/or middle region of the surface of the body of the patient 17 is therefore depicted for the user 28.

The activation state is depicted (visualized) by the depiction unit 34, in particular the projector. For this purpose a corresponding graphics symbol 37 is depicted (FIGS. 3 and 4) on the surface of the body of the patient 17. There are different graphics symbols 37 available for the different activation states of the activation status controller 33. The different graphics symbols 37 each include a circle, wherein for the different activation states of the respective circles are identified by a different color of an area of the respective circle. This is shown in FIG. 3 and FIG. 4 where one of the two different activation states is projected onto the patient 17 in each case. FIG. 4 visually depicts the second activation state in which the gesture detector is in non-detection mode. This activation state is depicted by the color red and symbolizes the second activation state to the user 28. FIG. 3 visually depicts the first activation state in which the gesture detector 32 is in a detection mode. This activation state is depicted by the color green and symbolizes the first activation state to the user 28.

To detect an input gesture of the user 28 a change in the activation state of the activation status controller 33 is necessary. In this connection the activation status controller 33 must change from the second activation state, in particular from the non-detection state, into the first activation state, in particular into the detection state. This change in the activation state of the activation status controller 33 is triggered by the user 28.

For this purpose, the user 28 can perform an activation gesture, causing an activation signal to be detected by the gesture detector 32, and this triggers the first activation state and thereby brings about a change in the activation status controller 33 from the second activation state into the first activation state. The activation gesture is a clearly defined gesture and it is only by recognition of this defined activation gesture that the change in the activation status controller 33 from the second activation state into the first activation state is triggered. In the exemplary embodiment the activation gesture is a hand 38 of the user 28 being placed on the graphics symbol, in particular on the area of the circle marked with red, which symbolizes the second activation state for the user 28. Any other activation gesture that is considered expedient to those skilled in the art is possible. Alternatively, the user 28 can trigger an activation signal by actuating the pedal, and thereby bring about a change in the activation status controller 33 from the second activation state into the first activation state.

Once the activation status controller 33 has been changed into the first activation state, input gestures and/or control gestures of the user 28 can be detected by the gesture detector 32. After activating detection of the input gestures and/or control gestures in a subsequent method step 101, input gestures and/or control gestures of the user 28 are detected. The input gesture and/or control gesture can be a gesture for controlling the magnetic resonance device and/or a gesture for inputting information and/or parameters for the magnetic resonance examination.

This method facilitates control of the medical imaging apparatus 10 during a medical imaging examination of the examination room 29. Activating gesture recognition is intuitive for the user 28 and enables quick and immediate detection of a current activation state of the activation status controller 33, so the user 28 can clearly see at any time whether gesture recognition is active or not. Furthermore, this method enables simple activation of gesture recognition for the user 28. In this way workflows for controlling the medical imaging examination can be optimized since, for example a physician who is carrying out an interventional examination on the patient 17, can control the medical imaging examination directly within the examination room 19. Furthermore, misinterpretations of input gestures for controlling the medical imaging apparatus 10 can be reduced and/or prevented.

Depiction of the symbols for displaying the current activation state of the activation status controller 33 can be adjusted by the activation status controller 33 to an examination region of the patient 17, so a size and/or spatial arrangement of the depicted symbols can also vary.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for operating a medical imaging apparatus comprising a medical data acquisition scanner operated by a control computer, said method comprising:
with a gesture detector in communication with said control computer, detecting at least one input gesture made by a user and providing an electronic signal, in response to said at least one input gesture, to said control computer that causes said control computer to control an aspect of operation of said data acquisition scanner dependent on said at least one input gesture; and
activating detection of said at least one input gesture by said gesture detector by providing an activation signal to said gesture detector from an activation status controller in response to a manual input to said activation status controller made by the user, with said gesture detector being enabled to detect said at least one input gesture only after being provided with said activation signal.

2. A method as claimed in claim 1 wherein said activation status controller has at least one activation state, and presenting a visual depiction of said at least one activation state to the user.

3. A method as claimed in claim 2 comprising visually presenting said depiction of said at least one activation state in a field of view of the data acquisition scanner in which medical data are acquired by the medical data acquisition scanner.

4. A method as claimed in claim 2 wherein said activation status controller has at least two different activation states, and visually depicting the different activation states with respective visual depictions selected from the group consisting of different symbols and different colors.

5. A method as claimed in claim 1 comprising configuring said gesture detector, when not activated to detect said at least one input gesture, to still be enabled to detect a gesture representing said manual input to said activation status controller, and detecting said gesture representing said manual input to said activation status controller with said gesture detector and thereafter activating said gesture detector from said activation status controller, to be enabled to detect said at least one input gesture.

6. A method as claimed in claim 1 comprising providing said manual input to said activation status controller from a manually operable actuator upon manual actuation of said manually operable actuator by said user.

7. A method as claimed in claim 1 comprising situating said data acquisition scanner and said gesture detector in an examination area, in which said medical data acquisition scanner is operated to acquire medical data, and situating said control computer in a control area that is physically separated from said examination area.

8. A medical imaging apparatus comprising:
a medical data acquisition scanner;
a control computer configured to operate said medical data acquisition scanner;
a gesture detector in communication with said control computer, said gesture detector being configured to detect at least one input gesture made by a user and to provide an electronic signal, in response to said at least one input gesture, to said control computer that causes said control computer to control an aspect of operation of said data acquisition scanner dependent on said at least one input gesture; and
an activation status controller configured to activate detection of said at least one input gesture by said gesture detector by providing an activation signal to said gesture detector from the activation status controller in response to a manual input to said activation status controller made by the user, with said gesture detector being enabled to detect said at least one input gesture only after being provided with said activation signal.

9. An apparatus as claimed in claim 8 wherein said activation status controller has at least one activation state, and comprising a presentation unit that causes a visual depiction of said at least one activation state to be presented to the user.

10. An apparatus as claimed in claim 9 wherein said presentation unit visually presents said depiction of said at least one activation state in a field of view of the data acquisition scanner in which medical data are acquired by the medical data acquisition scanner.

11. An apparatus as claimed in claim 9 wherein said activation status controller has at least two different activation states, and wherein said presentation unit visually depicts the different activation states with respective visual depictions selected from the group consisting of different symbols and different colors.

12. An apparatus as claimed in claim 8 wherein said gesture detector is configured, when not activated to detect said at least one input gesture, to still be enabled to detect a gesture representing said manual input to said activation status controller, and to detect said gesture representing said manual input to said activation status controller with said gesture detector and thereafter activating said gesture detector from said activation status controller, to be enabled to detect said at least one input gesture.

13. An apparatus as claimed in claim 8 comprising a manually operable actuator that provides said manual input to said activation status controller upon manual actuation of said manually operable actuator by said user.

14. An apparatus as claimed in claim 8 wherein said data acquisition scanner and said gesture detector are situated in an examination area, in which said medical data acquisition scanner is operated to acquire medical data, and said control computer is situated in a control area that is physically separated from said examination area.

15. An apparatus as claimed in claim 8 wherein said gesture detector comprises at least one camera.

16. A non-transitory, computer-readable data storage medium encoded with programming instructions, said storage medium being loaded into a control computer of a medical imaging apparatus that comprises a medical data acquisition scanner operated by the control computer, and a gesture detector, and an activation status controller, said programming instructions collectively causing said control computer, said activation status controller and said gesture detector to:
with said gesture detector, detect at least one input gesture made by a user and provide an electronic signal, in response to said at least one input gesture, to said control computer that causes said control computer to control an aspect of operation of said data acquisition scanner dependent on said at least one input gesture; and
activate detection of said at least one input gesture by said gesture detector by providing an activation signal to said gesture detector from an activation status controller in response to a manual input to said activation status controller made by the user, with said gesture detector being enabled to detect said at least one input gesture only after being provided with said activation signal.
